(19) United States
(12) Patent Application Publication
(10) Pub. No.: US 2023/0293093 A1
(43) Pub. Date: Sep. 21, 2023

(54) POSTURE DECISION APPARATUS, POSTURE DECISION METHOD AND PROGRAM

(71) Applicant: NIPPON TELEGRAPH AND TELEPHONE CORPORATION, Tokyo (JP)

(72) Inventor: Arinobu NIIJIMA, Tokyo (JP)

(73) Assignee: NIPPON TELEGRAPH AND TELEPHONE CORPORATION, Chiyoda-ku (JP)

(21) Appl. No.: 18/023,072

(22) PCT Filed: Aug. 27, 2020
(86) PCT No.: PCT/JP2020/032488

§ 371 Date: Feb. 24, 2023

(51) Int. Cl. A61B 5/00; G06F 3/16

(52) U.S. Cl. CPC A61B 5/4561; G06F 3/167; A61B 5/486; A61B 5/7405

(57) ABSTRACT

A posture determination device includes a sound wave reception unit configured to receive sound waves in an inaudible range output from a sound wave transmission unit worn by a user; and a posture determination unit configured to determine quality of a posture of the user based on the sound wave in the inaudible range and output a determination result.

### TECHNICAL FIELD

The present invention relates to a technology for correcting a bad posture of a user who is working.

### BACKGROUND ART

As opportunities for work or study using laptop PCs or smartphones increase, computer vision syndrome and text neck syndrome are becoming serious problems. When someone continues work with bad posture for a long time, eyestrain, eye dryness, neck pain, backaches, and the like are caused.

In order to prevent these symptoms, it is important for users to be conscious of good posture during work. For example, it is recommended that a distance of 40 cm or more between the eyes of a user and a screen is recommended when a user operates a laptop PC. When a user operates a smartphone, it is recommended that the user hold up the smartphone in front of their face and not operate the smartphone while looking down.

However, it is difficult for users to always be conscious of their posture during work. Therefore, a wearable sensor that always monitors such a posture has been developed, for example, Non Patent Literatures 1 and 2.

### CITATION LIST

### Non Patent Literature

[NPL 1] KHURANA, Rushil, et al. NeckGraffe: a postural awareness system. In: CHI′14 Extended Abstracts on Human Factors in Computing Systems. 2014. p. 227 to 232
[NPL 2] Min, Chulhong, et al. Tiger: Wearable Glass for the 20-20-20 Rule to Alleviate Computer Vision Syndrome. In: Proceedings of the 21st International Conference on Human-Computer Interaction with Mobile Devices and Services. 2019 p. 1 to 11.

### SUMMARY OF INVENTION

### Technical Problem

In the technology disclosed in NPL 1, a posture of a user is detected using an acceleration element (gyro). When it is determined that the detected posture is bad, a state of the posture is fed back to the user by using “vibration.” In the technology disclosed in NPL 1, an acceleration element (gyro) is adhered to the body of a user. However, it is difficult to adhere the element to the body to the user as a daily use device.

In the technology disclosed in NPL 2, a glasses type dedicated module is used, and the module is equipped with an inertia measuring device (IMU) that measures an angular velocity and acceleration with high accuracy. Since such a device is not a general-purpose device and is expensive, it is difficult for a user to use the device daily.

It is necessary to continuously use a device used to correct a bad posture during a user's work daily. However, in the above-described technology of the related art, it is necessary to mount a dedicated wearable device on a daily basis only for posture detection, and it is difficult to continuously use the wearable device because a load on a user is large.

The present invention has been made in view of the foregoing circumstances and an objective of the present invention is to provide a device that corrects a posture so that a user can continuously use the device with ease.

### Solution to Problem

According to the disclosed technology, a posture determination device includes:
a sound wave reception unit configured to receive sound waves in an inaudible range output from a sound wave transmission unit worn by a user; and
a posture determination unit configured to determine quality of a posture of the user based on the sound waves in the inaudible range and output a determination result.

### Advantageous Effects of Invention

According to the disclosed technology, it is possible to provide a device that corrects a posture and that a user can continuously use with ease.

### BRIEF DESCRIPTION OF DRAWINGS

FIG. 1 is a diagram illustrating an exemplary configuration of a posture feedback device.

FIG. 2 is a flowchart illustrating an operation of the posture feedback device.

FIG. 3 is a diagram illustrating an example of a specific device included in the posture feedback device.

FIG. 4 is a diagram illustrating an image of a positional relationship between a wearable speaker and a laptop PC.

FIG. 5 is a diagram illustrating an image of a positional relationship between a wearable speaker and a smartphone.

FIG. 6 is a diagram illustrating a power measurement result of a microphone of the laptop PC.

FIG. 7 is a diagram illustrating a power measurement result of a microphone of a smartphone.

FIG. 8 is a diagram illustrating an exemplary hardware configuration of a device.

### DESCRIPTION OF EMBODIMENTS

Hereinafter, an embodiment of the present invention (the present embodiment) will be described with reference to the drawings. Embodiments to be described below are merely exemplary, and the embodiments to which the present invention is applied are not limited to the following embodiments.

### Overview of Embodiment

In the present embodiment, a posture feedback device preventing computer vision syndrome and text neck syndrome using a wearable speaker (a neck speaker) that can be continuously used in a daily life without a load is provided.

In the posture feedback device according to the present embodiment, music is played from the wearable speaker and ultrasonic waves are simultaneously transmitted, the ultrasonic waves are picked up by a microphone of a laptop PC or a smartphone, a relative positional relationship between the wearable speaker and the microphone is estimated from a change in sound pressure, and a posture of a user is estimated from the relative positional relationship. When it is determined that the posture is bad, the sound quality is lowered by applying a sound effect to the playing music, and thus the user can be aware of the bad posture.

Since the posture feedback device according to the present embodiment is realized using a wearable speaker used for enjoying music in daily life, a wearing load on the user is small and the wearable speaker can be continuously used.

In the present embodiment, a wearable speaker (a neck speaker) is used as a sound wave transmission unit, but the wearable speaker (the neck speaker) is used as an example. Any device may be used as the sound wave transmission unit as long as the device is mounted on the upper body (a part above the waist) of the user and can transmit sound waves in an inaudible range so that a posture of the user can be determined.

For example, instead of the wearable speaker (the neck speaker), a headset (including an earphone) mounted on the head of the user may be used. Further, a speaker or a speakerphone mounted on the chest (in a breast pocket or the like) of the user may be used.

The head of the user, the shoulder of the user, the neck of the user, the chest of the user, and the like are all examples of the upper body of the user.

Hereinafter, a configuration and an operation of the posture feedback device according to the present embodiment will be described in detail with reference to the drawings.

(Configuration of Device)

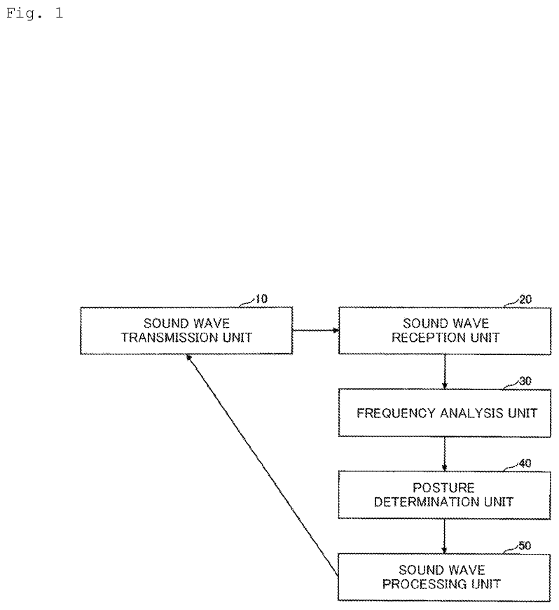

FIG. 1 illustrates an exemplary configuration of the posture feedback device according to the present embodiment. As shown in FIG. 1, the posture feedback device according to the present embodiment includes a sound wave transmission unit 10, a sound wave reception unit 20, a frequency analysis unit 30, a posture determination unit 40, and a sound wave processing unit 50. The functions of each unit are as follows. Since the posture feedback device is a device that determines whether the posture is good or bad, the device may be referred to as a posture determination device.

A device that does not include the sound wave transmission unit 10 and the sound wave processing unit 50 and includes the sound wave reception unit 20, the frequency analysis unit 30, and the posture determination unit 40 may be referred to as a posture determination device. The frequency analysis unit 30 may be included in the posture determination unit 40.

The sound wave transmission unit 10 simultaneously presents sound waves in an audible range and ultrasonic waves which are sound waves in an inaudible range. For convenience, the sound waves in the audible range may be referred to as a “sound.” The sound wave reception unit 20 receives the sound and the ultrasonic waves transmitted from the sound wave transmission unit 10. The frequency analysis unit 30 calculates a power spectrum by performing frequency analysis on a waveform of the signal of the sound waves received by the sound wave reception unit 20 through an FFT.

The posture determination unit 40 determines quality of a posture of the user based on the power of the ultrasonic waves estimated from the power spectrum calculated by the frequency analysis unit 30. The sound wave processing unit 50 processes the sound in the audible range transmitted from the sound wave transmission unit 10 in accordance with a posture determination result of the posture determination unit 40.

(Overview of Operation)

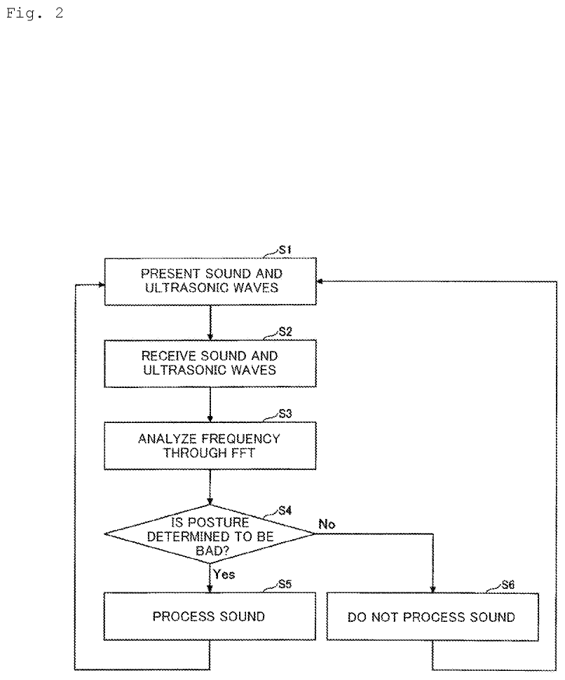

First, an overview of an operation of the posture feedback device according to the present embodiment will be described with reference to the flowchart of FIG. 2.

In S1 (step 1), the sound wave transmission unit 10 simultaneously transmits a sound and ultrasonic waves. In S2, the sound wave reception unit 20 receives the sound and the ultrasonic waves transmitted from the sound wave transmission unit 10.

In S3, the frequency analysis unit 30 samples the signal (the sound and the ultrasonic waves) received by the sound wave reception unit 20 and calculates a power spectrum by performing frequency analysis by an FFT for each given number of samples.

Thereafter, in S4, the posture determination unit 40 determines the quality of the posture of the user from the power in a frequency band of the ultrasonic waves and outputs the determination result. In S4, when the determination result indicating that the posture of the user is bad is output, the processing proceeds to S5. The sound wave processing unit 50 receiving the determination result processes the sound in the audible range and presents the processed sound (and ultrasonic waves) to the sound wave transmission unit 10. Thus, the user can be aware that her or his posture is bad. When it is determined in S4 that the posture of the user is not bad, the processing proceeds to S6 and returns to S1 without processing the sound.

As described above, as an example, the fact that the posture of the user is bad is fed back to the user by using the sound wave processing unit 50. The posture determination unit 40 may output information (for example, a vocal sound, light, an image, text, or the like) for feeding the fact that the posture of the user is bad to the user without using the sound wave processing unit 50.

(Specific Exemplary Configuration)

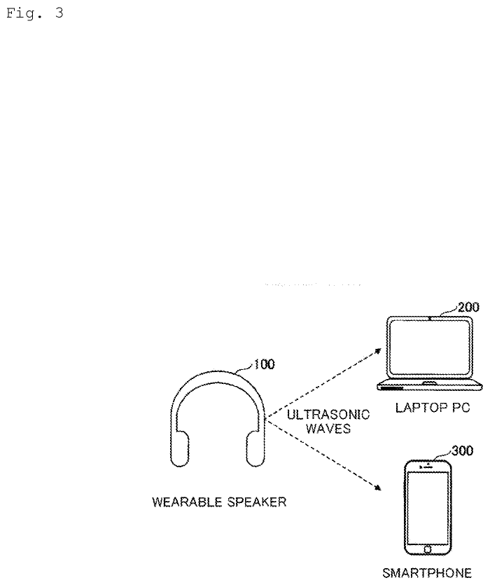

Hereinafter, the present embodiment will be described in more detail. FIG. 3 is a diagram illustrating an example in which the posture feedback device according to the present embodiment is mounted. In the present embodiment, a wearable speaker (a neck speaker) 100 is used as the sound wave transmission unit 10 that transmits a sound and ultrasonic waves. A laptop PC 200 or a smartphone 300 is used as a device that realizes the sound wave reception unit 20, the frequency analysis unit 30, the posture determination unit 40, and the sound wave processing unit 50.

More specifically, a microphone of the laptop PC 200 or the smartphone 300 corresponds to the sound wave reception unit 20. The frequency analysis unit 30, the posture determination unit 40, and the sound wave processing unit 50 are each realized by a program (an application) operating on the laptop PC 200 or the smartphone 300.

(Exemplary Operation)

Next, processing in the procedure of the flowchart shown in FIG. 2 will be described in more detail based on an example of the configuration in which the wearable speaker 100 and the laptop PC 200 or the smartphone 300 are used. Here, it is assumed that a wearable speaker 100 (a neck speaker), which is a sound wave transmission unit 10, is hung on the neck of a user or is worn near the shoulder of the user, and the user performs work with a laptop PC 200 or a smartphone 300.

<S1>

In S1, the sound wave transmission unit 10 (the wearable speaker 100) simultaneously transmits a sound and ultrasonic waves. The sound is a sound of music or the like heard by the user. A frequency of the ultrasonic waves is, for example, 20 kHz. By setting the frequency to 20 kHz, it is possible to present the ultrasonic waves which cannot be heard by the user and can be captured by the sound wave reception unit 20 (the microphone)

As an example, the sound wave transmission unit 10 (the wearable speaker 100) simultaneously transmits the sound and the ultrasonic waves. In a normal state (a state in which the posture of the user is not bad), only the ultrasonic waves may be transmitted without transmitting the sound. In this case, when the user is notified that her or his posture is bad, the sound wave transmission unit 10 (the wearable speaker 100) simultaneously transmits the sound.

Also, in either case of the normal state (the state in which the posture of the user is not bad) or the state in which the posture of the user is bad, the sound wave transmission unit 10 (the wearable speaker 100) may transmit only the ultrasonic waves. In this case, when the user is notified that the posture of the user is bad, for example, the posture determination unit 40 outputs information (for example, a vocal sound, light, an image, text, or the like) for feeding the fact that the posture of the user is bad back to the user from a display or a speaker of the laptop PC 200 or the smartphone 300.

<S2 and S3>

In S2, the sound wave reception unit 20 (the microphone) receives the sound and the ultrasonic waves transmitted from the sound wave transmission unit 10 (the wearable speaker 100).

In S3, the frequency analysis unit 30 samples a signal (the sound and the ultrasonic waves) received by the sound wave reception unit 20 (the microphone), calculates a power spectrum by performing frequency analysis through an FFT, for example, for every 1024 samples, and calculates power near 20 kHz which is the frequency of the ultrasonic waves. As will be described below, when a specific smartphone is used as a device that includes a microphone (the sound wave reception unit 20), power near 12 kHz may be calculated and power near 12 kHz may be used to determine the posture.

<S4>

In S4, the posture determination unit 40 determines whether the posture of the user is bad or not by a predetermined procedure based on the power near 20 kHz calculated by the frequency analysis unit 30. The details of the determination method will be described later.

<S5>

When it is determined in S4 that the posture of the user is bad, the processing proceeds to S5. The sound wave processing unit 50 processes the sound and causes the sound wave transmission unit 10 to transmit the processed sound. The ultrasonic waves are continuously transmitted from the sound wave transmission unit 10.

Although the sound processing method is not limited to a specific method, for example, the sound quality is reduced by applying an audio digital effect, for example, by adding white noise to the sound, applying a band-pass filter to narrow a sound range, or applying distortion to distort the sound. The user can be aware that her or his posture is bad from the deterioration in the sound quality and correct her or his posture in order to improve the sound quality.

(Example of Posture Determination Procedure)

An example of a posture determination procedure performed by the posture determination unit 40 when ultrasonic waves are presented downward (including an oblique downward direction) from the sound wave transmission unit 10 (the wearable speaker 100) will be described. Hereinafter, a case in which the user wearing the wearable speaker 100 operates the laptop PC 200 will be described as a first example and a case in which the user wearing the wearable speaker 100 operates the smartphone 300 will be described as a second example.

### First Example

When a posture of the user is bad (in a forward bent state) in a state in which the user is operating the laptop PC 200, a distance between the wearable speaker 100 and the microphone (the sound wave reception unit 20) of the laptop PC 200 becomes short. As a result, power (a sound pressure) of the ultrasonic waves increases.

Accordingly, when it is detected that the power of the ultrasonic waves becomes larger than a predetermined threshold, the posture determination unit 40 determines that the posture of the user operating the laptop PC 200 has become bad.

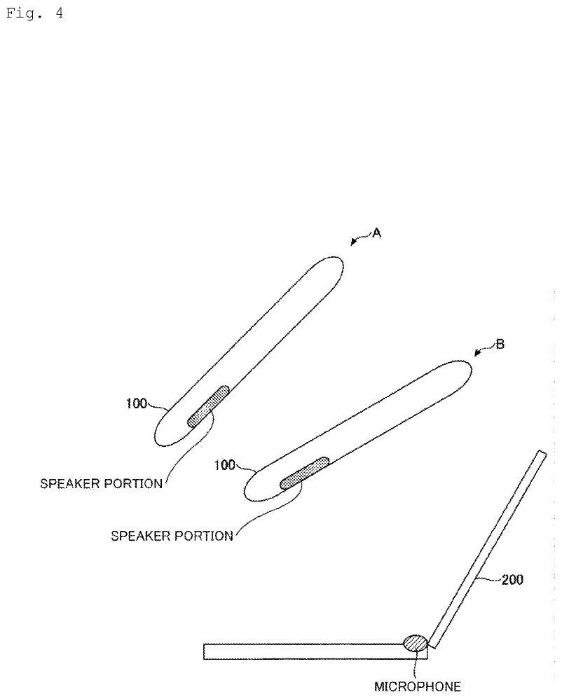

FIG. 4 is a diagram illustrating an image of the first example. In FIG. 4, a position of the wearable speaker 100 in a case in which the user is in the normal state is illustrated as A and a position of the wearable speaker 100 in a case in which the posture of the user has become bad is illustrated as B. As illustrated in FIG. 4, when A transitions to B, a speaker portion of the wearable speaker 100 (a portion from which the sound and the ultrasonic waves are output) is near the microphone. Therefore, the power of the ultrasonic waves increases, as described above.

### Second Example

When a posture of the user becomes bad in a state in which the user is operating the smartphone 300 (operating in a head-down state), the smartphone 300 is positioned below the wearable speaker 100, and thus the microphone (the sound wave reception unit 20) can easily pick up the ultrasonic waves. As a result, the power (the sound pressure) increases.

Accordingly, when it is detected that the power of the ultrasonic waves becomes larger than the predetermined threshold, the posture determination unit 40 determines that the posture of the user operating the smartphone 300 has become bad.

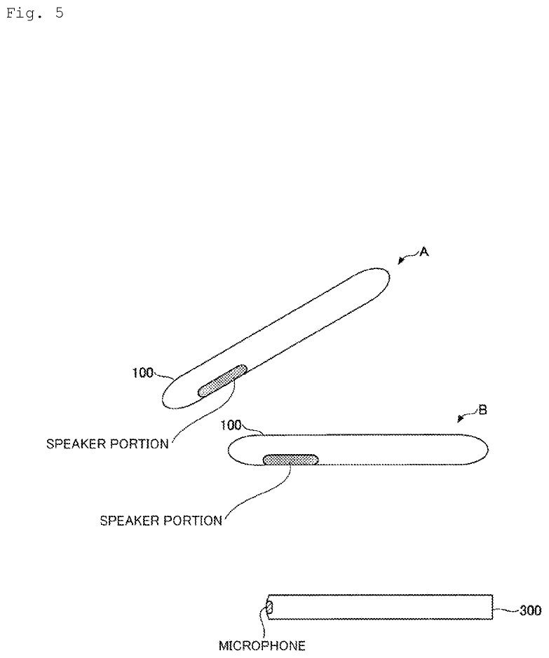

FIG. 5 is a diagram illustrating an image of the second example. In FIG. 5, a position of the wearable speaker 100 in a case in which the user is in the normal state is illustrated as A and a position of the wearable speaker 100 in a case in which the posture of the user has become bad is illustrated as B. As shown in FIG. 5, when A transitions to B, the smartphone 300 is positioned below the wearable speaker 100, and thus the power of the ultrasonic wave increases, as described above.

In either case of the first and second examples, the threshold can be determined by performing calibration (by measuring the power when the posture is good and bad) in advance.

(Experiment Results)

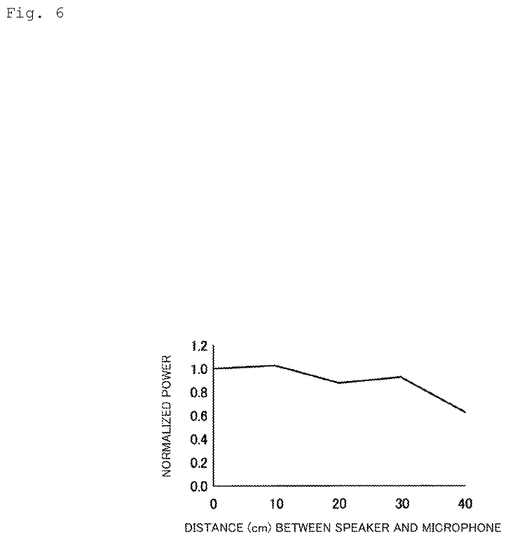

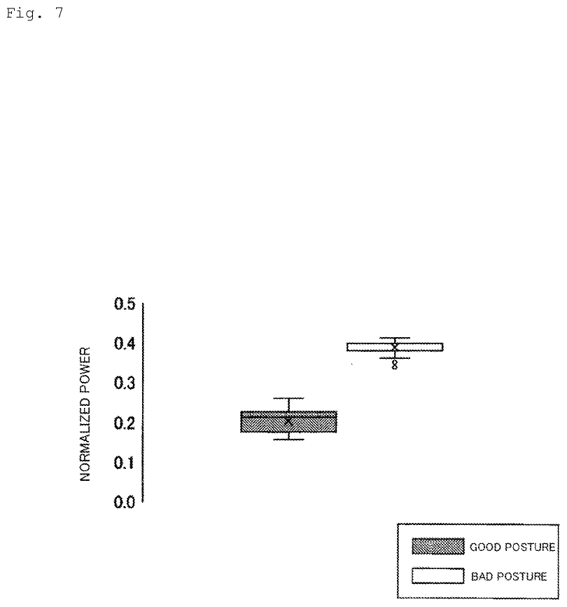

Actually, an experiment desired to face wearable speaker 100 downward, output the ultrasonic waves, and measure the power (the sound pressure) of the ultrasonic waves received by the microphone was conducted. Graphs of experiment results are illustrated in FIGS. 6 and 7.

FIG. 6 illustrates a value of a power spectrum near 20 kHz when a distance between the wearable speaker 100 and the microphone of the laptop PC 200 is changed from 0 cm to 40 cm at intervals of 10 cm.

Values of the graph indicate medians of the results measured 100 times at each distance and are normalized by dividing the values by the power at the distance of 0 cm. From FIG. 6, it is understood that the power increases more at the bad posture (0 to 30 cm), than at the good posture (40 cm).

FIG. 7 illustrates power when the user wearing the wearable speaker 100 is holding the smartphone 300 at a good posture (holding in front of her or his face) and when the user is holding the smartphone 300 at a bad posture (holding in a head-down state).

The results of the experiment are illustrated as box-plotted diagrams of results measured 100 times. In this case, normalization is performed with a value at the distance of 0 cm. From FIG. 7, it is understood that the power increases at the bad posture more than the good posture.

From the foregoing results, it can be understood that it is possible to estimate that the posture becomes bad in a state in which the user is operating the laptop PC 200 or the smartphone 300 by looking at a change in the power near 20 kHz. Further, since the ultrasonic waves do not interfere with the sound waves in the audible range, the wearable speaker 100 can be used while playing music. When the posture is bad, the user can be urged to improve her or his posture by lowering sound quality of the music.

The microphones of some smartphones have lowpass filters near 15 kHz in accordance with sound bands. In this case, power of 20 kHz cannot be calculated. However, it is known that a peak of power is seen as noise in an audible range (near 12 kHz) by ultrasonic waves because of characteristics of an amplifier of the microphone. Accordingly, when the laptop PC 200 or the smartphone 300 other than a specific smartphone is used, a posture may be determined with power near 20 kHz. When the specific smartphone is used, a posture may be determined with power near 12 kHz.

(Exemplary Hardware Configuration)

In the posture feedback device according to the present embodiment, the frequency analysis unit 30, the posture determination unit 40, and the sound wave processing unit 50 can all be realized, for example, by causing a computer including the microphone (the sound wave reception unit 20) to execute a program. The above-described laptop PC 200 and smartphone 300 are examples of the computer.

That is, the device (the device including, for example, the frequency analysis unit 30, the posture determination unit 40, and the sound wave processing unit 50) can be realized by executing a program corresponding to the processing executed by the device using hardware resources such as a CPU and a memory built in a computer. The program can be recorded on a computer-readable recording medium (a portable memory or the like) to be stored and distributed. The program can also be provided via a network such as the Internet or an electronic mail.

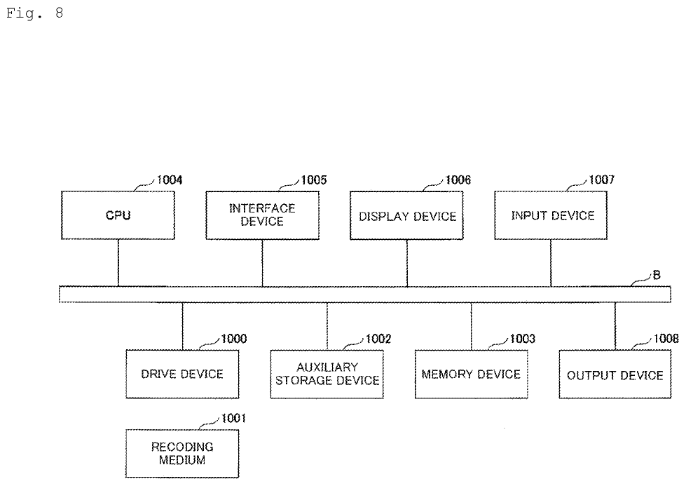

FIG. 8 is a diagram illustrating an exemplary hardware configuration of the computer. The computer in FIG. 8 includes a drive device 1000, an auxiliary storage device 1002, a memory device 1003, a CPU 1004, an interface device 1005, a display device 1006, an input device 1007, an output device 1008 which are connected to each other via a bus B.

A program realizing processing in the computer is provided by, for example, a recording medium 1001 such as a CD-ROM or a memory card. When the recording medium 1001 storing the program is set in the drive device 1000, the program is installed in the auxiliary storage device 1002 from the recording medium 1001 via the drive device 1000. However, the program may not necessarily be installed from the recording medium 1001 and may be downloaded from another computer via a network. The auxiliary storage device 1002 stores the installed program and also stores necessary files, data, and the like.

The memory device 1003 reads and stores the program from the auxiliary storage device 1002 when an instruction to start the program is given. The CPU 1004 implements a function related to the device in accordance with the program stored in the memory device 1003. The interface device 1005 is used as an interface for connection to a network. The display device 1006 displays a graphical user interface (GUI) or the like according to a program. The input device 1007 is configured with a keyboard, a mouse, buttons, a touch panel, and the like and is used to input various kinds of information. The output device 1008 outputs computation results.

### ADVANTAGEOUS EFFECTS OF EMBODIMENT

As described above, in the technology according to the present embodiment, it is possible to provide a device that corrects a posture and a user can continuously use with ease.

A wearable speaker used for enjoying music in a daily life is used as the sound wave transmission unit 10, and thus a configuration that a user can continuously use the sound wave transmission unit 10 with a small wearing load is provided. The sound wave transmission unit 10 outputting a sound and ultrasonic waves is not limited to a wearable speaker. A general-purpose device outputting a sound and ultrasonic waves is worn on a user, thereby providing a device that corrects a posture and the user can continuously use with ease.

As described above, in the technology disclosed in NPL 1, since the acceleration element (gyro) is adhered to the body of the user, it becomes difficult to use as a daily use device. On the other hand, in the technology according to the present embodiment, since a posture of the user can be detected by using ultrasonic waves and a state of the posture can be fed back to the user with a “sound.” Therefore, it is not necessary adhere to the body of the user and the user can continuously use the device according to the present embodiment in a daily life with ease.

In the technology disclosed in NPL 2, a dedicated device is used. It is difficult to use the dedicated device that is expensive and is used by the user daily. On the other hand, in the technology according to the present embodiment, a general-purpose device (for example, a wearable speaker) can be used as an ultrasonic presentation device and a feedback device for feeding a posture determination result back to a user. Therefore, the device configuration can be realized at low cost without using a dedicated device.

### CONCLUSION OF EMBODIMENT

The present specification discloses, at least, a posture determination device, a posture determination method, and a program according to each of the following clauses.

(Clause 1)

A posture determination device including:
a sound wave reception unit configured to receive sound waves in an inaudible range output from a sound wave transmission unit worn by a user; and
a posture determination unit configured to determine quality of a posture of the user based on the sound wave in the inaudible range and output a determination result.

(Clause 2)

The posture determination device according to Clause 1,
wherein the posture determination device includes the sound wave transmission unit, and
wherein the sound wave transmission unit is worn on an upper body of the user and transmits the sound wave of the inaudible range in a downward direction of the body of the user.

(Clause 3)

The posture determination device according to Clause 1 or 2, further including:
a frequency analysis unit configured to perform frequency analysis on the sound wave received from the sound wave reception unit and acquire power of the frequency of the sound wave in the inaudible range

(Clause 4)

The posture determination device according to Clause 3,
wherein the posture determination unit determines the quality of the posture of the user by comparing the power with a threshold.

(Clause 5)

The posture determination device according to any one of Clauses 1 to 4 further including:
a sound wave processing unit configured to process sound waves in the audible range output from the sound wave transmission unit in accordance with a posture determination result for the user.

(Clause 6)

The posture determination device according to Clause 5, wherein, when the posture determination unit determines that the posture of the user is bad, the sound wave processing unit lowers sound quality by applying an audio digital effect to the sound wave in the audible range.

(Clause 7)

A posture determination method executed by a posture determination device, the method including:
a sound wave reception step of receiving sound waves in an inaudible range output from a sound wave transmission unit worn by a user; and
a posture determination step of determining quality of a posture of the user based on the sound wave in the inaudible range and outputting a determination result.

(Clause 8)

A program causing a computer to function as a posture determination unit of the posture determination device according to any one of claims 1 to 6.

Although the embodiment has been described above, the present invention is not limited to such a specific embodiment, and various modifications and changes can be made within the scope of the gist of the present invention described in the claims.

### REFERENCE SIGNS LIST

10 Sound wave transmission unit
20 Sound wave reception unit
30 Frequency analysis unit
40 Posture determination unit
50 Sound wave processing unit
100 Wearable speaker
200 laptop PC
300 Smartphone
1000 Drive device
1001 Recording medium
1002 Auxiliary storage device
1003 Memory device
1004 CPU
1005 Interface device
1006 Display device
1007 Input device

1. A posture determination device comprising:
a sound wave receptor configured to receive sound waves in an inaudible range output from a sound wave transmitter worn by a user; and
a posture determiner configured to determine quality of a posture of the user based on the sound waves in the inaudible range and output a determination result.

2. The posture determination device according to claim 1, further comprising:
the sound wave transmitter, wherein the sound wave transmitter is adapted to be worn on an upper body of the user and is configured to transmit the sound waves of the inaudible range in a downward direction of the body of the user.

3. The posture determination device according to claim 1, further comprising:
a frequency analyzer configured to perform frequency analysis on the sound waves received from the sound wave receptor and acquire power of the frequency of the sound waves in the inaudible range.

4. The posture determination device according to claim 3, wherein the posture determiner determines the quality of the posture of the user by comparing the power with a threshold.

5. The posture determination device according to claim 1, further comprising:
a sound wave processor configured to process the sound waves in the audible range output from the sound wave transmitter in accordance with a posture determination result for the user.

6. The posture determination device according to claim 5, wherein, when the posture determiner determines that the posture of the user is bad, the sound wave processor lowers sound quality by applying an audio digital effect to the sound waves in the audible range.

7. A posture determination method executed by a posture determination device, the method comprising:
receiving sound waves in an inaudible range output from a sound wave transmitter worn by a user; and
determining quality of a posture of the user based on the sound waves in the inaudible range and outputting a determination result.

8. A computer-readable non-transitory recording medium storing computer-executable program instructions, that when executed by a processor causes a computer to function as the posture determination device according to claim 1.

9. The computer-readable non-transitory recording medium of claim according to claim 8, wherein the instructions when executed by the processor further cause the computer to transmit the sound waves of the inaudible range in a downward direction of the body of the user.

10. The non-transitory computer-readable medium according to claim 8, wherein the instructions when executed by the processor further cause the computer to perform frequency analysis on the sound waves and acquire power of the frequency of the sound waves in the inaudible range.

11. The non-transitory computer-readable medium according to claim 10, wherein the instructions when executed by the processor further cause the computer to determine the quality of the posture of the user by comparing the power with a threshold.

12. The non-transitory computer-readable medium according to claim 8, wherein the instructions when executed by the processor further cause the computer to process the sound waves in the audible range output in accordance with a posture determination result for the user.

13. The non-transitory computer-readable medium according to claim 12, wherein the instructions when executed by the processor further cause the computer to apply an audio digital effect to the sound waves in the audible range to lower sound quality when the posture of the user is determined to be bad.

14. The posture determination method of claim 7, wherein the sound wave transmitter is adapted to be worn on an upper body of the user and is configured to transmit the sound waves of the inaudible range in a downward direction of the body of the user.

15. The posture determination method of claim 7, further comprising performing frequency analysis on the sound waves and acquire power of the frequency of the sound waves in the inaudible range.

16. The posture determination method of claim 15, further comprising determining the quality of the posture of the user by comparing the power with a threshold.

17. The posture determination method of claim 7, further comprising processing the sound waves in the audible range output in accordance with a posture determination result for the user.

18. The posture determination method of claim 17, further comprising applying an audio digital effect to the sound waves in the audible range to lower the sound quality when the posture of the user is determined to be bad.
